(19) United States
(12) Patent Application Publication
(10) Pub. No.: US 2023/0173444 A1
(43) Pub. Date: Jun. 8, 2023

(54) PROCESSES FOR REDUCING THE RATE OF PRESSURE DROP INCREASE IN A VESSEL

(71) Applicant: Dow Technology Investments LLC, Midland, MI (US)

(72) Inventors: Daniel Grohol, Midland, MI (US); David G. Barton, Midland, MI (US)

(21) Appl. No.: 17/997,716

(22) PCT Filed: Jun. 2, 2021
(86) PCT No.: PCT/US2021/035337

§ 371 Date: Nov. 1, 2022

Related U.S. Application Data
(60) Provisional application No. 63/046,350, filed on Jun. 30, 2020.

(51) Int. Cl. B01J 8/00

(52) U.S. Cl. CPC B01J 8/0015; B01J 2208/00539; C07C 29/141

(57) ABSTRACT

The present invention generally relates to processes for reducing the rate of pressure drop increase in a vessel used for hydrogenation of aldehydes to alcohols. In one embodiment, the process comprises replacing a first set of catalyst pellets with a second set of catalyst pellets, wherein the second set of catalyst pellets have a higher average aspect ratio than the first set of catalyst pellets, a different shape than the first set of catalyst pellets, or a combination thereof, and wherein a void fraction of the second set of catalyst pellets is greater than the void fraction of the first set of catalyst pellets, wherein a pressure drop rate increase of the vessel partially filled with the second set of catalyst pellets is less than a pressure drop rate increase of the vessel partially filled with the first set of catalyst pellets when operated under substantially similar conditions.

### FIELD

The present invention relates to processes for reducing the rate of pressure drop increase in a vessel and, in particular, to processes for reducing the rate of pressure drop increase in a vessel used for the hydrogenation of aldehydes to alcohols.

### BACKGROUND

The use of heterogeneous (packed bed) hydrogenation catalysts for the reduction of aldehydes to the corresponding alcohols is well known. A number of issues need to be considered in such hydrogenation processes including, for example, reactivity, selectivity (avoiding side-reactions), and pressure drop across the bed, especially in connection with vapor-phase hydroformylation processes. In general, a low and steady pressure drop across the bed is preferred. Considerable work has been done in connection with the shape and size of the hydrogenation catalyst pellets to address these issues.

However, as a catalyst is used, it has been found that whatever the shape of the starting catalyst pellet, the solid catalyst pellet degrades to generate “fines” or “catalyst dust.” The exact nature of these “fines” is undefined and may vary depending on the nature of the catalyst and the pellet but the ability of the catalyst bed to tolerate the generation of these fines has not been addressed in the past. A catalyst bed that starts off with excellent performance but degrades rapidly (i.e., exhibits rapid pressure drop increase with time) will need to be replaced frequently which requires a plant shutdown and expensive catalyst recovery or disposal.

It would be desirable to have processes to reduce the rate of pressure drop increase in a vessel while minimizing the impact on catalyst performance.

### SUMMARY

The present invention relates to processes for reducing the rate of pressure drop increase in a vessel used for hydrogenation of aldehydes to alcohols. In embodiments of the present invention, a first set of catalysts pellets is replaced with a second set of catalyst pellets having a higher aspect ratio than the first set of catalyst pellets and a higher void fraction. By increasing the void fraction, such processes advantageously increase the catalyst bed life as it has been found that increasing the void fraction increases the time it takes for the bed to fill with catalyst fines. Replacing the catalyst pellets with catalyst pellets having a higher aspect ratio (i.e., lengthening the primary axis of the catalyst pellets) provides an increase in void fraction. There are practical limitations to increasing the pellet length and aspect ratio: A longer pellet may be susceptible to premature breaking, undesirably forming additional fines.

In the hydrogenation of aldehydes to alcohols, a vessel (e.g., a reactor) is used. The vessel has an inlet and an outlet, and is partially filled with a first set of catalyst pellets. A majority of the catalyst pellets comprise a catalytic metal. The catalyst pellets each have an aspect ratio and a shape. The first set of catalyst pellets has a void fraction. When the vessel is partially filled with the first set of catalyst pellets, the vessel exhibits a pressure drop increase rate (i.e., the rate at which the pressure drop across the vessel increases over time).

In an embodiment of the present invention, the process comprises replacing the first set of catalyst pellets with a second set of catalyst pellets, wherein the second set of catalyst pellets have a higher average aspect ratio than the first set of catalyst pellets, a different shape than the first set of catalyst pellets, or a combination thereof, and wherein a void fraction of the second set of catalyst pellets is greater than the void fraction of the first set of catalyst pellets, wherein a pressure drop rate increase of the vessel partially filled with the second set of catalyst pellets is less than a pressure drop rate increase of the vessel partially filled with the first set of catalyst pellets when operated under substantially similar conditions.

These and other embodiments are discussed in more detail in the Detailed Description below.

### BRIEF DESCRIPTION OF THE DRAWINGS

FIG. 1 is a schematic that illustrates how void space in a fixed bed within a vessel increases with increasing aspect ratio of a catalyst pellet.

FIG. 2 is a plot illustrating change in pressure drop relative to air flow as discussed in the Examples section.

FIG. 3 is a plot illustrating pressure drop increase as a consequence of fines as discussed in the Examples section.

### DETAILED DESCRIPTION

All references to the Periodic Table of the Elements and the various groups therein are to the version published in the CRC Handbook of Chemistry and Physics, 72nd Ed. (1991-1992) CRC Press, at page I-11.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc.

As used herein, the term “ppmw” means parts per million by weight.

As used herein, the term “hydrogenation” is contemplated to include, but is not limited to, all hydrogenation processes that involve converting one or more substituted or unsubstituted aldehyde compounds or a reaction mixture comprising one or more substituted or unsubstituted aldehyde compounds to one or more substituted or unsubstituted alcohols or to a reaction mixture comprising one or more substituted or unsubstituted alcohols. The alcohols may be asymmetric or non-asymmetric. In some embodiments, the starting aldehyde may be unsaturated (conjugated or not conjugated with the aldehyde moiety) and the resulting product may be the corresponding saturated or unsaturated alcohol. Embodiments of the present invention are particularly useful in vapor phase hydrogenation processes, especially the hydrogenation of aldehydes to alcohols.

In the hydrogenation of aldehydes to alcohols, a vessel (e.g., a reactor) is used. The vessel has an inlet and an outlet, and is partially filled with a first set of catalyst pellets. A majority of the catalyst pellets comprise a catalytic metal. The catalyst pellets each have an aspect ratio and a shape. The first set of catalyst pellets has a void fraction. When the vessel is partially filled with the first set of catalyst pellets, the vessel exhibits a pressure drop increase rate (i.e., the rate at which the pressure drop across the vessel increases over time).

In an embodiment of the present invention, the process comprises replacing the first set of catalyst pellets with a second set of catalyst pellets, wherein the second set of catalyst pellets have a higher average aspect ratio than the first set of catalyst pellets, a different shape than the first set of catalyst pellets, or a combination thereof, and wherein a void fraction of the second set of catalyst pellets is greater than the void fraction of the first set of catalyst pellets, wherein a pressure drop rate increase of the vessel partially filled with the second set of catalyst pellets is less than a pressure drop rate increase of the vessel partially filled with the first set of catalyst pellets when operated under substantially similar conditions. In some embodiments, the second set of catalyst pellets differs from the first set of catalyst pellets only in having a higher average aspect ratio than the first set of catalyst pellets.

When comparing the pressure drop rate increase of the vessel between the second set of catalyst pellets and the first set of catalyst pellets, the comparison is based on operating the process at substantially similar conditions. In other words, the catalyst bed temperature and pressure and the gas flow rates through the vessel containing the second set of catalyst pellets are maintained substantially similar (within experimental error) to those of the vessel containing the first set of catalyst pellets.

In some embodiments, the average aspect ratio of the second set of catalyst pellets is 50% to 300% greater than the average aspect ratio of the first set of catalyst pellets. In some embodiments, the void fraction of the second set of catalyst pellets is at least five percent larger than the void fraction of the first set of catalyst pellets. In some embodiments, the void fraction of the second set of catalyst pellets is at least nine percent larger than the void fraction of the first set of catalyst pellets. In some embodiments, the void fraction of the second set of catalyst pellets is up to thirty percent larger than the void fraction of the first set of catalyst pellets. In some embodiments, the void fraction of the second set of catalyst pellets is up to twenty percent larger than the void fraction of the first set of catalyst pellets. In some embodiments, the void fraction of the second set of catalyst pellets is from five to thirty percent larger than the void fraction of the first set of catalyst pellets. In some embodiments, the void fraction of the second set of catalyst pellets is from five to twenty percent larger than the void fraction of the first set of catalyst pellets.

In some embodiments, the second set of catalyst pellets provide a catalyst life that is longer than a catalyst life provided by the first set of catalyst pellets when operated under substantially similar conditions.

In some embodiments, the second set of catalyst pellets comprise the same catalytic metal and the same catalyst support as the first set of catalyst pellets.

Hydrogen and one or more aldehydes are the reactants used in the hydrogenation for which processes of the present invention are directed. Hydrogen may be obtained from any suitable source including, without limitation, petroleum cracking and refinery operations. The one or more aldehydes are typically obtained from hydroformylation of olefins (which themselves may be obtained from any suitable source including, without limitation, petroleum cracking and refinery operations), aldol condensation of aldehydes, or other processes known to those of ordinary skill in the art such as ozonation of olefins, acetal or hemi-acetal hydrolysis, or fermentation or other bio-sources.

The aldehyde starting materials can be non-optically active aldehydes and/or optically active aldehydes. Illustrative non-optically active aldehyde starting materials include e.g., propionaldehyde, n-butyraldehyde, isobutyraldehyde, n-valeraldehyde, 2-methyl 1-butyraldehyde, hexanal, hydroxyhexanal, 2-methyl valeraldehyde, heptanal, 2-methyl 1-hexanal, octanal, 2-methyl 1-heptanal, nonanal, 2-methyl-1-octanal, 2-ethyl 1-heptanal, 3-propyl 1-hexanal, decanal, adipaldehyde, 2-methylglutaraldehyde, 2-methyladipaldehyde, 3-methyladipaldehyde, 3-hydroxypropionaldehyde, 6-hydroxyhexanal, alkenals, e.g., 2-, 3- and 4-pentenal, alkyl 5-formylvalerate, 2-methyl-1-nonanal, undecanal, 2-methyl 1-decanal, dodecanal, 2-methyl 1-undecanal, tridecanal, 2-methyl 1-tridecanal, 2-ethyl, 1-dodecanal, 3-propyl-1-undecanal, pentadecanal, 2-methyl-1-tetradecanal, hexadecanal, 2-methyl-1-pentadecanal, heptadecanal, 2-methyl-1-hexadecanal, octadecanal, 2-methyl-1-heptadecanal, nonodecanal, 2-methyl-1-octadecanal, 2-ethyl 1-heptadecanal, 3-propyl-1-hexadecanal, eicosanal, 2-methyl-1-nonadecanal, heneicosanal, 2-methyl-1-eicosanal, tricosanal, 2-methyl-1-docosanal, tetracosanal, 2-methyl-1-tricosanal, pentacosanal, 2-methyl-1-tetracosanal, 2-ethyl 1-tricosanal, 3-propyl-1-docosanal, heptacosanal, 2-methyl-1-octacosanal, nonacosanal, 2-methyl-1-octacosanal, hentriacontanal, 2-methyl-1-triacontanal, and the like. Illustrative optically active aldehyde starting materials include (enantiomeric) aldehyde compounds such as, e.g. S-2-(p-isobutylphenyl)-propionaldehyde, S-2-(6-methoxy-2-naphthyl)propionaldehyde, S-2-(3-benzoylphenyl)-propionaldehyde, S-2-(p-thienoylphenyl)propionaldehyde, S-2-(3-fluoro-4-phenyl)phenylpropionaldehyde, S-2-[4-(1,3-dihydro-1-oxo-2H-isoindol-2-yl)phenyl]propionaldehyde, and S-2-(2-methylacetaldehyde)-5-benzoylthiophene.

The nature and composition of aldehyde hydrogenation catalysts are well known. The hydrogenation catalyst used in hydrogenation processes are provided as pellets and referred to herein as catalyst pellets. The use of the word “pellet” is not intended to limit the pellet to a specific shape (e.g., cylindrical), and such pellets can be a variety of shapes as described further herein. In addition, not all catalyst pellets will include a catalytic metal but as set forth herein, a majority of the catalyst pellets used in hydrogenation processes of the present invention will include a catalytic metal. Thus, catalyst pellets useful in the hydrogenation process comprise a catalyst support, and a majority of the catalyst pellets in a vessel where hydrogenation occurs further comprise a catalytic metal. The catalytic metal can include Group 8, 9, and 10 metals selected from rhodium (Rh), cobalt (Co), copper (Cu), iridium (Ir), ruthenium (Ru), iron (Fe), nickel (Ni), palladium (Pd), platinum (Pt), chromium (Cr), osmium (Os) and mixtures thereof, with preferred metals being palladium, platinum, and nickel.

Catalyst supports are solid materials designed to hold the active catalyst metal. Examples include graphite, activated carbon, silica, alumina, and metal oxides such as molybdenum oxide, chromium oxide, zinc oxide, titania, and the like. Catalyst supports may be composed of a combination of different materials and other additives which offer different properties such as improved crush strength, reduced metal leaching, reduced side products, and ease of extrusion among others. The catalytic metal may be on the surface, in pores, and/or within the catalyst support itself. The chemical nature of the catalyst support is not narrowly critical for the present invention. In some embodiments, catalyst supports are generally inert.

Typical catalyst beds in hydrogenation reaction vessels also include additional catalyst pellets such as glass or ceramic beads typically at the top and/or bottom of the catalyst bed to aid gas distribution, and such catalyst pellet may not include a catalytic metal. Such catalyst pellets are generally not part of the active catalyst zone and generally do not contribute significantly to the pressure drop.

As used herein, in reference to a catalyst pellet, the term “aspect ratio” refers to the longest axis of the pellet divided by the average of the remaining axes. For example, if a catalyst pellet has three special axes (e.g., x, y, z), the aspect ratio is the longest axis divided by the average of the remaining two axes. The general shape of many catalyst pellets is a cylinder, spherocylinder, or otherwise similar to a cylinder such as twisted spirals, helical wound shapes, or lobed cylinders and the like (see, e.g., U.S. Pat. No. 467,366, U.S. Pat. No. 5,168,090, EP 2 886 194, and U.S. Pat. No. 10,005,079). For example, in a shape such as a twisted spiral, a general cylindrical shape can be seen such that at least two axes are essentially the same (the “diameter” of the spiral shape) and the third is uniquely different (the “length” of the spiral cylinder). In embodiments of the present invention utilizing such a catalyst pellet shape, it is the third uniquely different axis (the length or longitudinal axis) that is typically changed to effect a change in the aspect ratio. The “average aspect ratio” of a plurality of catalyst pellets in a vessel is the sum of the aspect ratios of the individual catalyst pellets divided by the number of catalyst pellets.

As used herein, the term “catalyst pellet fraction” is the volume occupied by the catalyst pellets. The sum of the “catalyst pellet fraction” and the “void fraction” (as defined below) constitutes the “total reaction space” and is the observed volume of a certain mass of catalyst pellet, often referred to as the packing density. With a known reactor geometry and the observed packed height, the “total reaction space” and packing density are calculated.

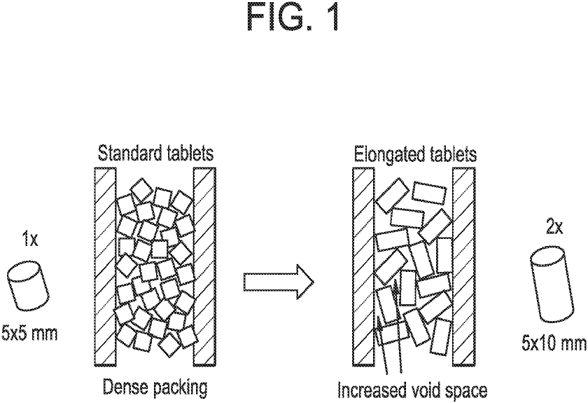

As used herein, the terms “void fraction” or “void fraction ratio” refer to the ratio of the volume of empty (non-catalyst pellet filled) space between and within catalyst pellets relative to the total reaction space. It is well known in the art that with heterogeneous, particle-based catalyst pellets, there will be spaces between the particles and these spaces exhibit and promote a random flow pattern (i.e., avoid straight paths or “channels”) to maximize internal mixing and uniform flow and optimal material distribution throughout the bed. The void space is different than the space within the particles such as in pores. FIG. 1 is a schematic that illustrates how void space in a fixed bed within a vessel increases with increasing aspect ratio of a catalyst pellet.

It should be noted that the exact composition and microscopic (pore) structure of the heterogeneous hydrogenation catalyst is not narrowly critical to the present invention which is dealing with the shape of the catalyst pellets as they pack in the catalyst bed. The nature of fines from the catalyst pellets, how they are generated, and how they migrate in the bed are also not narrowly critical other than they are observed and changes in pressure drop are observed to the degree that catalyst performance and/or catalyst bed performance is negatively impacted.

As noted above, in some embodiments, the second set of catalyst pellets comprise the same catalytic metal and the same catalyst support as the first set of catalyst pellets. That is, the second set of catalyst pellets, in such embodiments, differ only from the first set of catalyst pellets in aspect ratio and/or shape, but not from the perspective of chemical composition.

Analytical techniques for measuring catalytic metal concentrations are well known to the skilled person, and include atomic absorption (AA), inductively coupled plasma (ICP) and X-ray fluorescence (XRF). Unless otherwise noted herein, references to catalytic metal concentration are concentrations measured using X-ray fluorescence.

As an illustration, a catalytic metal may be impregnated onto any solid catalyst support, such as inorganic oxides, (i.e. alumina, silica, titania, or zirconia) carbon, or ion exchange resins. The catalytic metal may be supported on, or intercalated inside the pores of, a zeolite, glass or clay; the catalytic metal may also be dissolved in a liquid film coating the pores of said zeolite or glass. Such zeolite-supported catalytic metals are particularly advantageous for producing one or more regioisomeric alcohols in high selectivity, as determined by the pore size of the zeolite. The techniques for supporting catalytic metals on solids, such as incipient wetness, are well-known to those having ordinary skill in the art. The solid catalyst pellet thus formed may still be complexed with one or more of the ligands defined above. Descriptions of such solid catalyst pellets may be found in for example: J.Mol. Cat., 1991, 70, 363-368; Catal. Lett., 1991, 8, 209-214; J. Organomet. Chem., 1991, 403, 221-227; Nature, 1989, 339, 454-455; J. Catal., 1985, 96, 563-573; J. Mol. Cat., 1987, 39, 243-259.

Processes of the present invention comprise contacting at least one kind of aldehyde with hydrogen under heterogeneous vapor phase hydrogenation conditions sufficient to form at least one kind of alcohol product in a vessel. The vessel has an inlet, an outlet, and a volume. The vessel is partially filled with catalyst pellets, wherein a majority of the catalyst pellets comprise a catalytic metal.

Pressure drop as used herein is the difference in pressure between the inlet of the vessel (typically measured at or near the aldehyde feed point) and the outlet of the vessel. As the reaction fluid passes through the heterogeneous catalyst pellets, the fluid encounters resistance due to the catalyst pellets which results in a drop in pressure as the reaction fluid flows through the vessel. Excessive pressure drop can result in further catalyst pellet degradation (e.g., crushing or abrasion) and in the case of vapor phase hydrogenation, possible condensation, channeling, and heat transfer issues. Fine particles that are produced in this process tend to increase flow resistance and thus become a major contributor to pressure drop increase with time and can lead to the need to change out the catalyst pellets.

It is known that fines can be generated at the start of the catalyst life such as during the initial filling of the catalyst reactor. Over time, the generation of fines can cause a pressure drop rate increase. As used herein, the term “a pressure drop rate increase” refers to the length of time for the pressure drop to increase to a certain critical pressure drop value whether by a linear increase, an exponential increase, or sometimes by a sudden stepwise increase. A critical pressure drop value can be identified by persons having ordinary skill in the art based on the equipment being used in the process (e.g., vessel size, compressor size, etc.), the observation of condensation in the vessel, process economics (e.g., amount of product not aligned to costs of production), and other factors. The exact nature of the fines and how they are generated is often not known but are usually attributed to catalyst fracturing, crushing, abrasion, chemical/physical erosion (leaching) and the like.

The critical pressure drop value will differ depending on the catalyst system and equipment, of course, but when the catalyst reactor efficiency and operation is impacted by the pressure drop, it becomes an economic decision as to whether to continue at sub-optimal performance (e.g., lower rates, lower conversion, higher side products) or to stop operation and change out the catalyst pellets. The present invention extends the life of the catalyst pellets in a vessel and can advantageously delay and/or reduce the costs of a plant shutdown and catalytic metal recovery or disposal.

It is recognized that increasing the void volume reduces the packed density of a catalyst bed and historically this has been viewed as undesirable. However, although the total reactor volume may have to be slightly larger to accommodate the same mass of catalytic metal, the longer catalyst pellet life for the lifetime of a plant can more than justify any initial higher capital expense.

Some embodiments of the invention will now be described in more detail in the following Examples.

### EXAMPLES

All parts and percentages in the following examples are by weight unless otherwise indicated. Pressures are given as absolute pressure unless otherwise indicated.

Two batches of cylindrical catalyst pellets with average aspect ratios of 1 and 2 are pressed. The batch with an average aspect ratio of 1 is a cylinder with a diameter roughly equivalent to the height, and is referred to as the “1X Catalyst Pellets.” The batch with an average aspect ratio of 2 is an elongated cylinder with the height roughly twice as long as the diameter, and is referred to as the “2X Catalyst Pellets.” The particle densities are measured from the weights and dimensions of individual pellets. The bulk densities and void volumes of the pellets are then measured by filling a large tube of known dimensions with each batch of catalyst pellets. The void volumes are calculated as: 1-(bulk density/particle density). The void fraction of the 1X Catalyst Pellets is found to be 40.7% and that of the 2X Catalyst Pellets is found to be 44.5%.

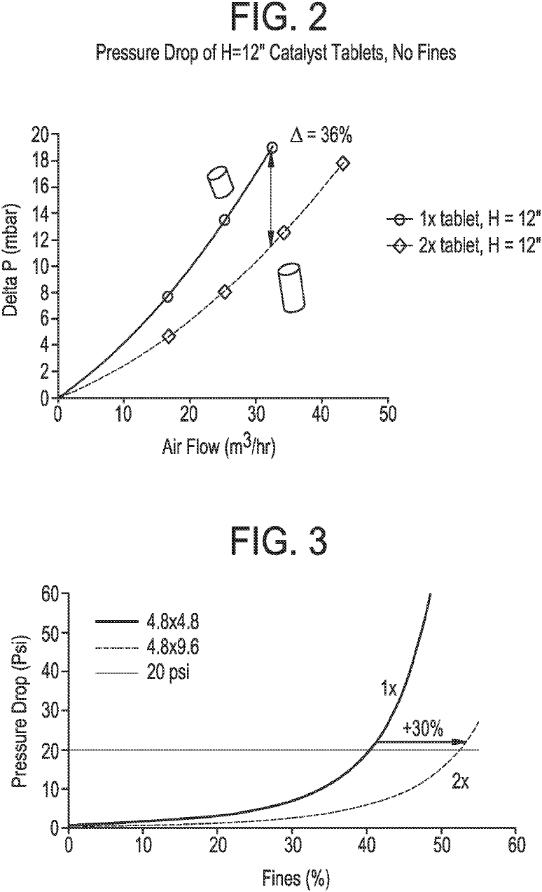

The pressure drops of 12 inch columns of each batch of catalyst pellets is then measured at varying flow rates using a pressure drop apparatus constructed for this purpose. The pressure drop apparatus includes a vertical tube having a 4-inch diameter and an approximately 18-inch length. The vertical tube has a metal screen at the bottom with openings smaller than the catalyst pellets held inside the tube. The bottom and the top of the tube are open to allow for forced airflow through the tube, with a blower located either upstream or downstream of the tube and with an airflow meter. A filter is also placed just downstream of the catalyst-holding vertical tube in order to remove any loose catalyst particles and dust exiting the catalyst tube area. The walls of the tube contain openings called pressure taps at two or more different heights that are connected with a tube to a pressure drop cell to monitor the pressure difference between these heights. The low-pressure tap is located at least 1 inch above the bottom screen holding the catalyst pellets and the top pressure tap is located at least an inch below the top level of the catalyst column in order to avoid the pressure drop anomalies associated with airflow entry and exit effects. The airflow through the column of catalyst pellets may be regulated either with different power settings on the blower or by a baffle placed into the airflow path constricting the airflow to a desired value. This method of control allows for the acquisition of pressure drop values for a charge of desired catalyst pellets at different airflow values. The data are plotted and shown in FIG. 2. The pressure drop of the 12″ column of the 2X Catalyst Pellets is found to be about 36% lower than the pressure drop of the 12″ column of the 1X Catalyst Pellets.

The addition of fines from catalyst pellets in vessels and their impact on pressure drop and catalyst life is carried out by Discrete Element Method (DEM) modeling (CD_adapco, Star-CCM+, Melville, NY: CE-Adapco, 2019). The simulations are done by randomly injecting particles at different percentages of fines in a cylindrical reactor (vessel) with a diameter (D) of 11 centimeters and a height (H) of 9 centimeters. Based on the percentage of fines generated, the catalyst pellet size is determined maintaining the same catalyst pellet aspect ratio as the initial catalyst pellet and a constant number of catalyst pellets (N) is injected into the container. N is the number of catalyst pellets with 0% fines that completely fills the cylindrical reactors which is found to 5500 and 2559 for the 1X Catalyst Pellets and 2X Catalyst Pellets, respectively. For each DEM simulation, the average height (H) of catalyst pellets is measured and the total volume occupied by the catalyst pellets is calculated (V_{c}= π/4(D²H)). Because the mass of the particles is assumed conserved (i.e., no fines enter or leave the reactor, the catalyst pellet volumes are constant (Vₛ= Nπ/4(dₚ²L)) where dₚ is the characteristic particle diameter. The void fraction is then calculated by equation 4: [symbol] Pressure drops are calculated using the Ergun method (as described in S. Ergun, “Fluid flow through packed columns,” Chem. Eng. Prog., Vol. 8, pp. 89-94 (1952)). The sphericity factor (ϕₛ) and the Ergun equation constants (C₁, C₂) are fit to match experimental data (0% fines) using the nonlinear least squares method.

The growth of pressure drop is plotted versus the amount of fines generated during catalyst aging as shown in FIG. 3. The maximum allowable pressure drop is set at a constant value of 20 psi. It is found that the 2X Catalyst Pellets would take about 30% more fines to reach the maximum allowable pressure drop. If the amount of fines formed from abrasion or erosion of the catalyst pellets build linearly with time, the use of the 2X Catalyst Pellets would result in about 30% longer life than for the 1X Catalyst Pellets due to any pressure drop limitations. Thus, replacing the 1X Catalyst Pellets with 2X Catalyst Pellets can reduce the rate of pressure drop increase in a vessel (such as a reactor for hydrogenation of aldehydes to alcohols) and increase the catalyst life.

1. A process for reducing the rate of pressure drop increase in a vessel used for hydrogenation of aldehydes to alcohols, wherein the vessel comprises an inlet and an outlet and has an empty volume, wherein the vessel is partially filled with catalyst pellets wherein a majority of the catalyst pellets comprise a catalytic metal, wherein when the vessel is partially filled with a first set of catalyst pellets, each having an aspect ratio and shape, the vessel exhibits a pressure drop increase rate which is the rate at which the pressure drop increases across the vessel over time, wherein the first set of catalyst pellets has a void fraction, the process comprising:
(a) replacing the first set of catalyst pellets with a second set of catalyst pellets, wherein the second set of catalyst pellets have a higher average aspect ratio than the first set of catalyst pellets, a different shape than the first set of catalyst pellets, or a combination thereof, wherein a void fraction of the second set of catalyst pellets is greater than the void fraction of the first set of catalyst pellets;
wherein a pressure drop rate increase of the vessel partially filled with the second set of catalyst pellets is less than a pressure drop rate increase of the vessel partially filled with the first set of catalyst pellets when operated under substantially similar conditions.

2. The process of claim 1, wherein the average aspect ratio of the second set of catalyst pellets is 50% to 300% greater than the average aspect ratio of the first set of catalyst pellets.

3. The process of claim 1, wherein the void fraction of the second set of catalyst pellets is at least five percent larger than the void fraction of the first set of catalyst pellets.

4. The process of claim 1, wherein the second set of catalyst pellets provide a catalyst life that is longer than a catalyst life provided by the first set of catalyst pellets when operated under substantially similar conditions.

5. The process of claim 1, wherein the second set of catalyst pellets comprise the same catalytic metal and the same catalyst support as the first set of catalyst pellets.
